(19) United States
(12) Patent Application Publication
(10) Pub. No.: US 2002/0122743 A1
(43) Pub. Date: Sep. 5, 2002

(54) Ultraviolet sterilization apparatus and method

(76) Inventor: Ruiyan Huang, Changsha (CN)

Correspondence Address: Squire, Sanders & Dempsey L.L.P, Two Renaissance Square, 40 North Central Avenue, Suite 2700, Phoenix, AZ, 85004-4498, US

(21) Appl. No.: 09/798,683

(22) Filed: Mar. 2, 2001

(51) Int. Cl. A61L 2/10

(52) U.S. Cl. 422/24

(57) ABSTRACT

A method and device for sterilizing an object in a dual-purpose household appliance such as microwave ovens, dishwashers, laundry machines, etc. The device includes a housing unit having a chamber for containing the object to be sterilized, an ultraviolet light source positioned outside the chamber and an exposure panel of a fixed, retractable, or flip open design, the exposure panel enabling the ultraviolet light source to emit ultraviolet radiation onto the object to be sterilized.

### BACKGROUND

1. Field of the Invention

The present invention relates to a method and apparatus for sterilizing objects using ultraviolet radiation.

2. Related Art

Due to the increase in awareness of the potentially harmful effects that microorganisms, such as fungi, bacteria and viruses can have on humans, there have been several attempts to provide devices that sterilize food, liquids and other items.

For example, (i) U.S. Pat. No. 5,886,329 to Kim generally discloses an antibiotic microwave oven that utilizes high frequency energy and an antibiotic surface to kill bacteria bread within the cooking chamber and prevent propagation of harmful microbes; (ii) U.S. Pat. No. 4,448,750 to Fuesting generally discloses a method for disinfecting or sterilizing small objects by placing them in an aqueous solution and exposing them to ultrasonic and ultraviolet radiation; (iii) A toothbrush conditioner, whereby toothbrushes are conditioned through exposure to ultraviolet radiation, is disclosed by U.S. Pat. No. 4,803,364 to Ritter; (iv) Contact lenses sterilized by exposure to variations of energy levels of a microwave field in a microwave oven are disclosed by U.S. Pat. No. 4,956,155 to Rohrer et al.; and (v) Food products sterilized by exposure to a radio frequency field are disclosed by U.S. Pat. No. 3,272,636 to Fehr et al. These and other attempts have previously been made to combat harmful microbes.

Among the various sterilization methods, ultraviolet radiation has proven to be effective in the harmful effects of unwanted microbes. When various microorganisms are exposed to ultraviolet radiation for a period of time, many of the microorganisms are killed and are therefore effectively neutralized.

Of the various systems and methods described in the above-cited references, many include multiple devices and/or complicated methods for sterilization. Moreover, if it is desired to sterilize, for example, contact lenses and a toothbrush, separate devices must be obtained for each item to be sterilized.

Boucher, in U.S. Pat. No. 3,926,556, which is incorporated herein by reference, discloses a method and apparatus for low temperature intermittent or continuous destruction of microorganisms for decontamination of organic fluids using the synergistic effects of combined ultraviolet radiation and microwave energy. Boucher's apparatus involves the use of an ultraviolet lamp inside a microwave chamber, wherein sterilization is obtained through the synergistic combination of non-thermal biocidal effects of microwaves and the biocidal effects of ultraviolet radiation. Boucher's device uses a microwave cavity having a series of parallel capillary glass tubes for liquid flow and ultraviolet lamps positioned next to the glass tubes. Boucher's device is very complicated and not suitably used in microwaves for home use, as the device does not allow use of a microwave cavity for cooking purposes, e.g., for temperatures over 100 degrees.

Le Vay discloses an invention in U.S. Pat. No. 5,166,528, which is also incorporated herein by reference, which basically converts Boucher's invention for application in microwave ovens for home use. Le Vay describes an ultraviolet sterilizer that includes a housing having ultraviolet lamps. A microwave field generated by the microwave oven activates the ultraviolet lamps. The device disclosed by Le Vay solves many of the previous problems with sterilizers, but requires a specially designed holder having multiple microwave-activated ultraviolet lamps. This device poses additional problems such as where to put the holder when not in use (i.e., during cooking); how to protect the ultraviolet lamps from being broken when the holder is being stored; and difficulties associated with cleaning the specially adapted holder. The devices disclosed by Boucher and Le Vay have limitations in that they cannot be used for sterilization of objects that are sensitive to microwaves (e.g., metals, fresh fruit or vegetables). While Boucher explicitly explains that biocidal effects are derived from non-thermal effects of microwaves, it is not disputed that the microwaves can generate a significant amount of heat in the objects to be sterilized.

Newman, in U.S. Pat. No. 6,165,526, which is incorporated herein by reference, discloses a microwave oven having ultraviolet lamps disposed in the oven chamber for decontamination of food. However, sterilization lamp is placed within the chamber, which decreases oven space, increases the difficulty of oven chamber cleaning, and increases the likelihood of potential damage to UV lamps by being impacted during cleaning and/or cooking and contamination by food particles.

### SUMMARY OF THE INVENTION

Accordingly, it is an object of the present invention to solve one or more of the foregoing problems. The invention includes an apparatus having (1) a housing unit including a chamber for containing an object to be sterilized, and (2) an ultraviolet light source disposed in the housing unit at a location outside of the chamber and operative to expose the object to be sterilized upon activation of a sterilization process.

In an exemplary implementation of the present invention, the housing unit is a microwave oven adapted to sterilize objects with an ultraviolet light source. The sterilization equipment is not present within the chamber during periods when the oven is used for cooking. When a sterilization process is activated, an ultraviolet light source is controlled to expose an object in the chamber to ultraviolet radiation for a predetermined period of time. Utilizing the present invention, special devices for sterilizing are not required, but rather are integrated with conventional features of a microwave oven. Additionally, chamber space is not reduced by sterilization equipment in the microwave oven.

In one embodiment of the invention, a window is provided between the sterilization chamber and the ultraviolet radiation source. The window may be reflective of microwaves on a chamber side and transparent to ultraviolet radiation on the source side.

In another embodiment of the invention, an openable window is provided to expose objects in the sterilization chamber during a sterilization process and closeable to protect the UV radiation source during non-sterilization periods.

In yet another embodiment of the invention, a pivoting door is provided in a wall of the sterilization chamber and when activated, the ultraviolet source moves through the pivoting door to enter the chamber and expose an object to be sterilized to UV radiation.

By providing a UV radiation source that is out-of-chamber during normal use of an appliance, a seamless transition is obtained between use of the device as traditional appliance and/or a sterilization device. Implementation of the invention in a microwave oven enables microwave-sensitive objects to be sterilized in an integrated package that is available for home use.

For materials that are not sensitive to microwaves, as disclosed by Boucher, a synergistic combination of microwave and ultraviolet radiations can be used to effectively sterilize an object when the ultraviolet sterilizer is implemented in a microwave oven. However, with the sterilizer of the present invention, effective sterilization can be achieved without the use of microwaves and the sterilizer according to the present invention does not consume any extra space in a microwave chamber. Implementation of the present invention in a microwave oven may be advantageous as a majority of microwave ovens include a rotating turntable that improves effectiveness of exposure of an object being sterilized to the ultraviolet radiation source.

According to various aspects of the present invention, chamber space is reserved for objects to be microwaved or sterilized when the ultraviolet radiation source is located outside of the appliance chamber. Additionally, the risk of contamination of the UV lamp surface is reduced when, for example, food being prepared in a microwave oven, hence effectiveness of sterilization is assured. According to further aspects of the present invention, disposing UV lamps outside of a sterilization chamber during periods of non-sterilization reduces the risks of accidentally breaking UV lamps of a sterilizer.

### BRIEF DESCRIPTION OF THE DRAWING

FIG. 1 is a perspective view of an ultraviolet sterilization device according to an exemplary embodiment of the present invention.

FIG. 2 is a cross sectional view of a wall of a sterilization chamber having an ultraviolet radiation source according to one embodiment of the present invention.

FIG. 3 is a cross sectional view of a sterilization chamber wall having an ultraviolet radiation source according to another embodiment of the present invention.

FIG. 4 illustrates a cross-sectional view of a sterilization chamber wall having an ultraviolet radiation source in a modified implementation of the present invention.

FIG. 5 illustrates a cross-sectional view of a sterilization apparatus during a non-sterilizing period according to another embodiment of the present invention.

FIG. 6 is a cross-sectional view of the sterilization apparatus of FIG. 5 when a sterilization process is activated.

FIG. 7 is a top view of the sterilization apparatus of FIG. 5.

FIG. 8 is a schematic diagram of a sterilization apparatus control device according to one implementation of the present invention.

FIG. 9 is a cross-sectional view of a sterilization apparatus according to another embodiment of the present invention.

FIG. 10 is a side view of the sterilization apparatus of FIG. 9.

### DETAILED DESCRIPTION OF THE PREFERRED EMBODIMENTS

As described with reference to the application drawing, like elements are designated with like reference characters.

According to an exemplary embodiment of the present invention, an ultraviolet sterilizer is provided that kills microorganisms using ultraviolet radiation. An ultraviolet sterilizer according to the present invention uses at least one UV lamp to produce ultraviolet radiation for any predetermined period of time, which may be selected by the user, and/or preprogrammed into the device, thereby destroying harmful microbes present on objects subjected to the ultraviolet radiation.

The ultraviolet sterilizer 100 includes a housing unit 110 having a chamber 120 for sterilizing objects. An object to be sterilized is placed in chamber 120. In a preferred embodiment, chamber 120 is a chamber of a common household device (e.g., microwave, toaster-oven, dishwasher, range-top oven, refrigerator, or crock pot). In the preferred embodiment, sterilizer 100 is a modified microwave oven and sterilization chamber 120 is a food-cooking chamber of the microwave oven.

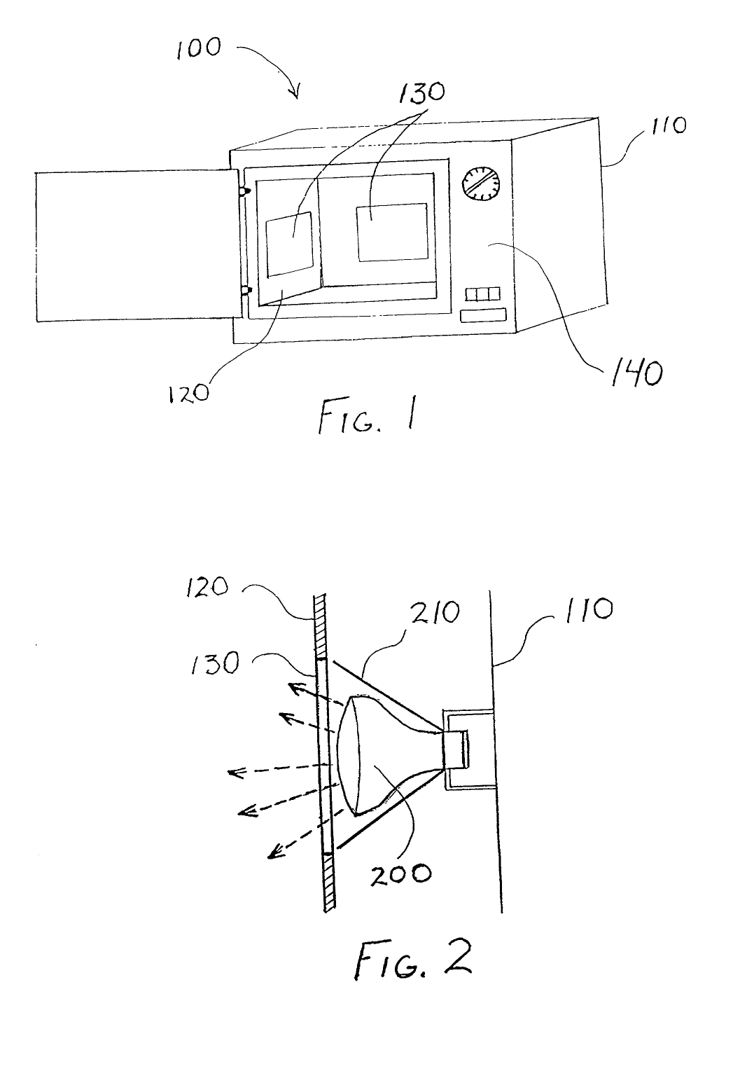

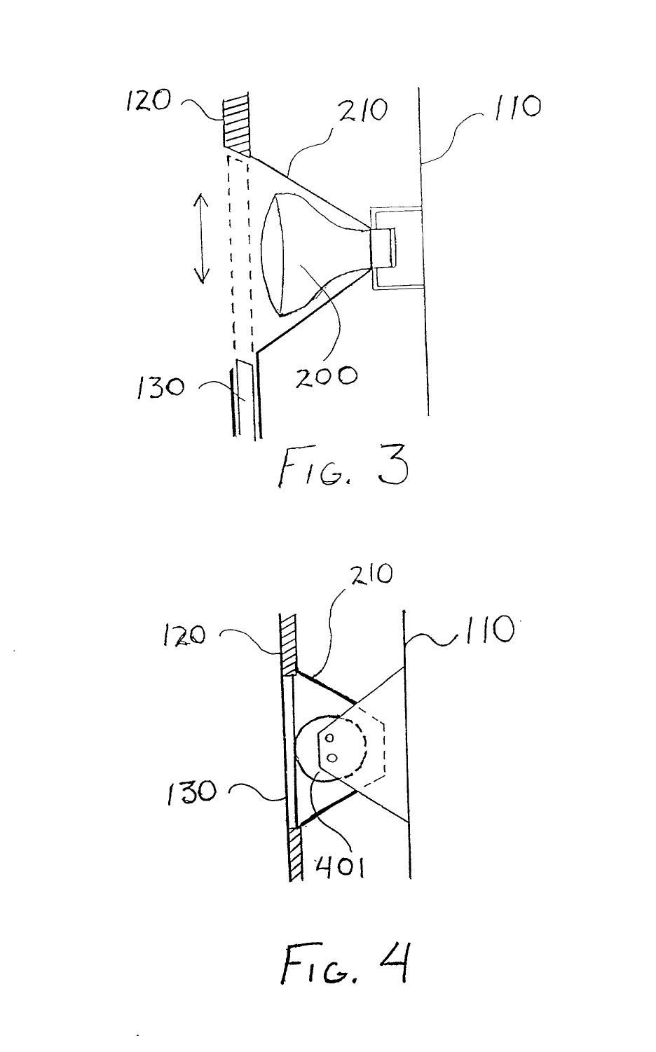

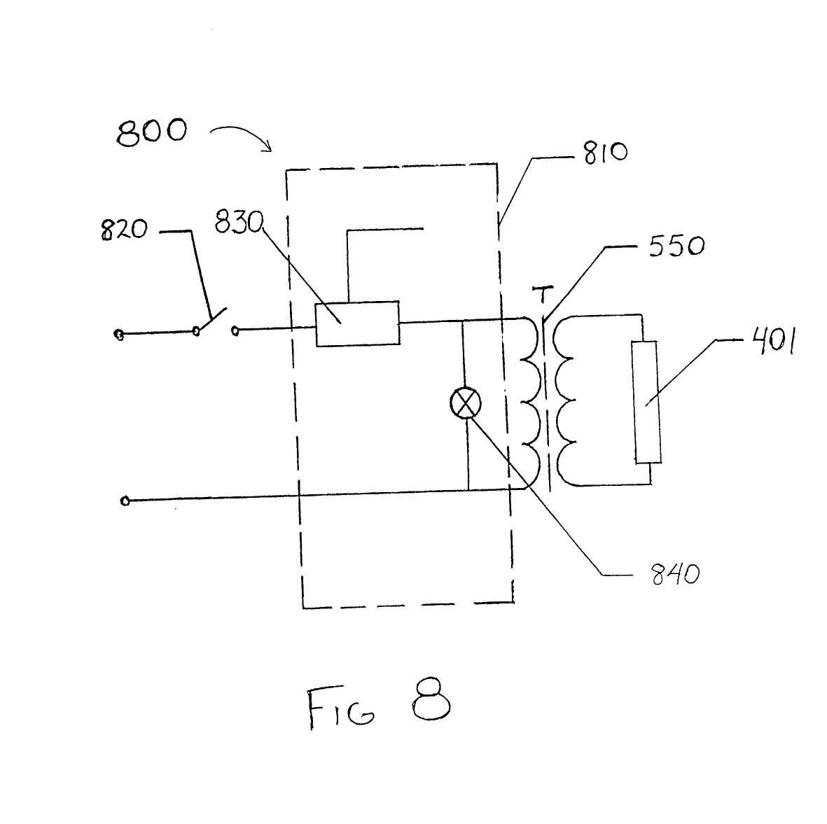

Once the object to be sterilized (hereinafter “object”) is placed in chamber 120, the sterilization process is activated by control panel 140, that also serves as control panel for setting cooking power and times in microwave oven 100. Upon activation of the sterilization process, the object is irradiated by a UV lamp (shown in FIGS. 2-10) for a predetermined period of time. Irradiation of the object occurs through an exposure panel 130 located on an interior wall of chamber 120. There are several different implementations for exposure panel 130 as described in further detail below.

As shown in the ultraviolet sterilizer 100 of FIG. 1, one or more exposure panels 130 may be located on any one as well as on all sides of the chamber 120. For maximum elimination of microorganisms on objects to be sterilized, it is preferable that all surfaces of the object be subjected to direct exposure of ultraviolet radiation. Therefore, positioning exposure panels 130 on the top, bottom and sides of the ultraviolet sterilizer 100 may promote improved sterilization.

The ultraviolet sterilizer of FIG. 1 may be used to kill harmful microorganisms on any number of household items. Some potential objects to be sterilized in the home may include, but are not limited to, dishes, silverware, food items, contact lenses, cell phones, pacifiers, baby bottles, toys, razors, dish rags and washcloths, toothbrushes, false teeth, tools, containers, fruits, vegetables, etc.

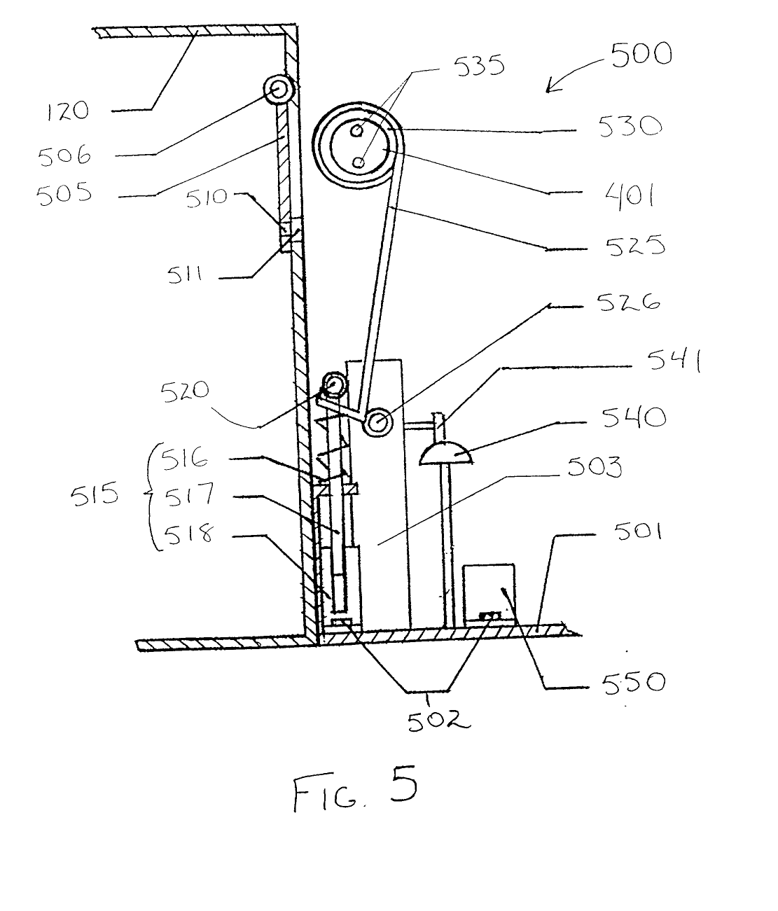

Turning now to FIG. 2, an example embodiment of exposure panel 130 is shown. Ultraviolet radiation source 200 is positioned between a wall of the chamber 120 and a wall of housing unit 110. In FIGS. 2-3, ultraviolet radiation source 200 is of a flood lamp design and is mounted to the wall housing unit with reflector 210 via a conventional socket unit. Exposure panel 130 in FIG. 2 is a fixed-window configuration made of a material that is substantially transparent to ultraviolet radiation on the UV radiation source 200 side of the chamber 120 (arrows represent example UV radiation penetration through panel 130). In the exemplary implementation of the invention using a microwave oven, the exposure panel material would preferably be reflective to microwave radiation on a chamber side of the panel 130. The fixed-window configuration of exposure panel 130 is preferable when implementing the sterilization device in water oriented appliances such as a dishwasher and/or laundry machine, since the exposure panel 130 would protect UV lamp 200 from exposure to water.

FIG. 3 illustrates an implementation of the invention that does not require exposure panel 130 to be transparent to ultraviolet radiation. Exposure panel 130 is configured to be open and closed during and after a sterilization process, respectively. Accordingly, exposure panel 130 may be made of an opaque material similar to that used in the chamber of a microwave oven; or in the case when sterilizer 100 is a conventional/toaster oven or refrigerator, a material may be selected that is temperature resistant and thus, protects ultraviolet source 200 when closed, e.g., during periods of non-sterilization. A mechanism (not shown) to open and close exposure panel 130 in the implementation of FIG. 3, can be any conventional means for electrically controlled mechanical systems such as a servo mechanism, a relay system, a pulley mechanism, etc. In this implementation of the present invention, the exposure panel 130 is opened during activation of a sterilization process, and closed during periods of nonsterilization. When operated in this manner, ultraviolet lamp 200 is protected from harmful effects generated by the device in which it is implemented (e.g., dishwasher, ovens, microwaves, laundry washer and dryers, etc.). It may also be advantageous for cleaning the sterilization chamber 120 when the exposure panels 130 of various embodiments of the present invention are substantially flush with an interior wall of the chamber 120.

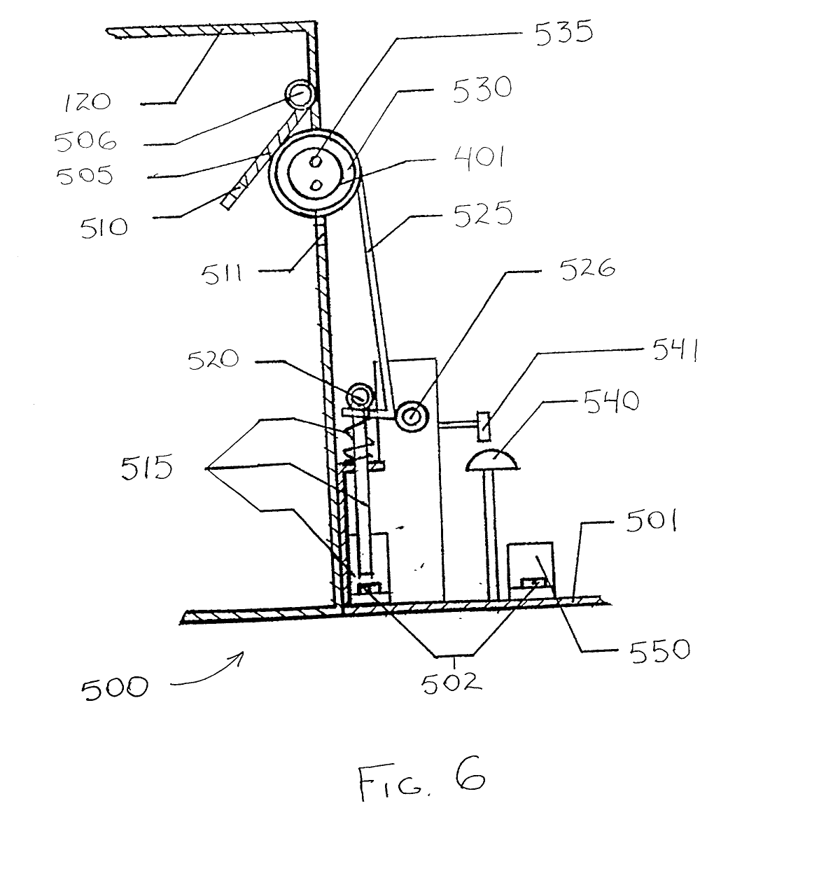

Turning to FIG. 4, an embodiment of the present invention using a cylindrical UV lamp 401 is shown. Cylindrical UV lamp 401 is an example implementation of the present invention similar to FIGS. 2-3, using a different variety of radiation source. Both types of ultraviolet radiation sources 200, 401 may have the same effectiveness in sterilizing objects, but may be selected because of design considerations such as available space, availability of the lamps, associated costs, electrical connectivity, durability, etc. It should be recognized that while a fixed, UV transparent exposure panel 130 is shown in FIG. 4, the exposure panel may also be retractable similar to the implementation of the invention in FIGS. 3 and 9-10, or a flip open window implementation (FIG. 5).

Generally, there are two types of UV lamps available (in all shapes and sizes): (i) one with high ozone content that causes harmful side effects such as headaches, vomiting, etc. when human tissue is irradiated directly; and (ii) one with low ozone content and harmless to the health of humans. In the latter case, the intensity of the lamp is selected on recommendation of the World Health Organization (WHO) and the Environmental Protection Agency (EPA). In one implementation of the present invention, a low-ozone quartz UV lamp 401 is selected having electrodes activated similar to fluorescent lights.

An example UV lamp that has been used in the present invention is a 15 Watt, 220 volt, ZSZ Low Ozone Quartz UV Sterilization Lamp made by Tianjing No. 5 Lamp Company. However, it should be recognized that any commercially available UV lamp may work in the present invention, the selection of which is significantly dependent on design considerations such as, power consumption, amount of time desired for sterilization, the effectiveness of sterilization desired, type of microorganism to be neutralized, etc.

When a ten watt UV lamp is used, an average time for eliminating 99.9% of E.coli and other viruses and bacteria, in a space of .128m³ at exposure of 20,000 uW/cm², is seven minutes. If the bulb wattage is changed to fifteen watts, sterilization will be obtained, with the same factors, in approximately four minutes.

The ultraviolet sterilizer discussed in the above implementations of the present invention utilizes a fixed UV lamp disposed in commonly used household appliances. However, it should be noted, that the inventor also contemplates that the ultraviolet sterilizer 100 can be implemented as a standalone device (e.g., without combination with other household appliances). Additionally, as shown by the FIG. 5 embodiment of the present invention and described below, the UV lamp is not required to be fixed.

FIGS. 5-7 shows another preferred embodiment of an ultraviolet sterilizer according to the present invention. As with all of the implementations of the present invention, the ultraviolet radiation source 401 is located outside the sterilization chamber 120. However, with the embodiment of FIG. 5, a radiation source mounting unit 500 enables UV lamp 401 to be inserted into chamber 120 through exposure panel 505 to perform sterilization. In this implementation, exposure panel 505 is a push-up or flip-open window configured to allow the UV lamp 401 to enter into the chamber 120. Exposure panel 505 is rotatably attached to a portion of the chamber 120 by a hinge 506 and a latching mechanism including magnets 510 and 511 to maintain a closed position during period of non-sterilization.

Radiation source mounting unit 500 is attached to base 501 of housing unit 110 adjacent to chamber 120, by fasteners 502. Radiation source mounting unit 500 is configured to move UV lamp 401 in and out of chamber 120 using a relay mechanism 515, which in this implementation, includes compressive spring 516, magnetic core 517 and electromagnet 518.

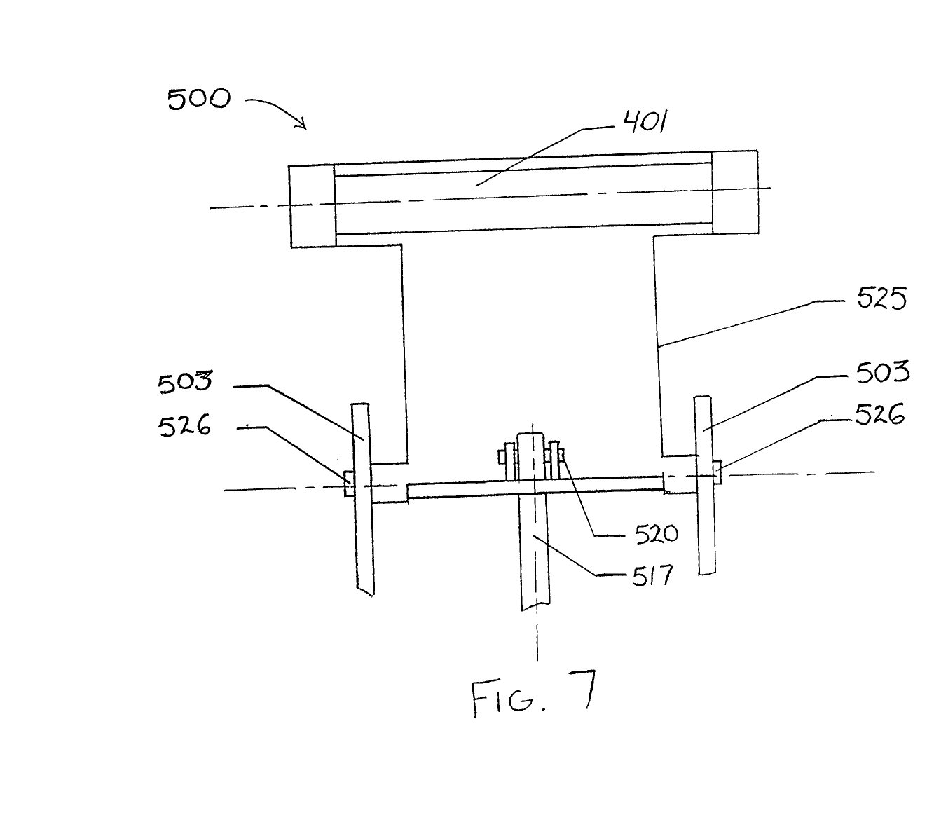

As shown in FIGS. 5-7, magnetic core 517 pivotally attaches to movable lamp arm 525 by a latch arm deadbolt 520. During activation of a sterilization process, electromagnet 518 is engaged causing movement of magnetic core 517 to a downward position, which in turn, rotates movable lamp arm 525 toward chamber 120 (see FIG. 6). A movable arm coupling 526 facilitates rotation of movable lamp arm 525.

An optional adaptation to radiation source mounting unit 500 is the inclusion of an alarm system that notifies a user when sterilization is complete. An alarm system according to one implementation of the invention shown in FIGS. 5-6 includes a bell 540, which is activated by a bell hammer 541 when movable lamp arm 525 returns to a non-sterilizing position (FIG. 6).

UV lamp 401 is mounted in a movable lamp arm 525 via a soft lamp base 530 and a lamp connector 535 that includes an L line conductor and an N line conductor. Magnetic leakage converter 550 is for providing AC power.

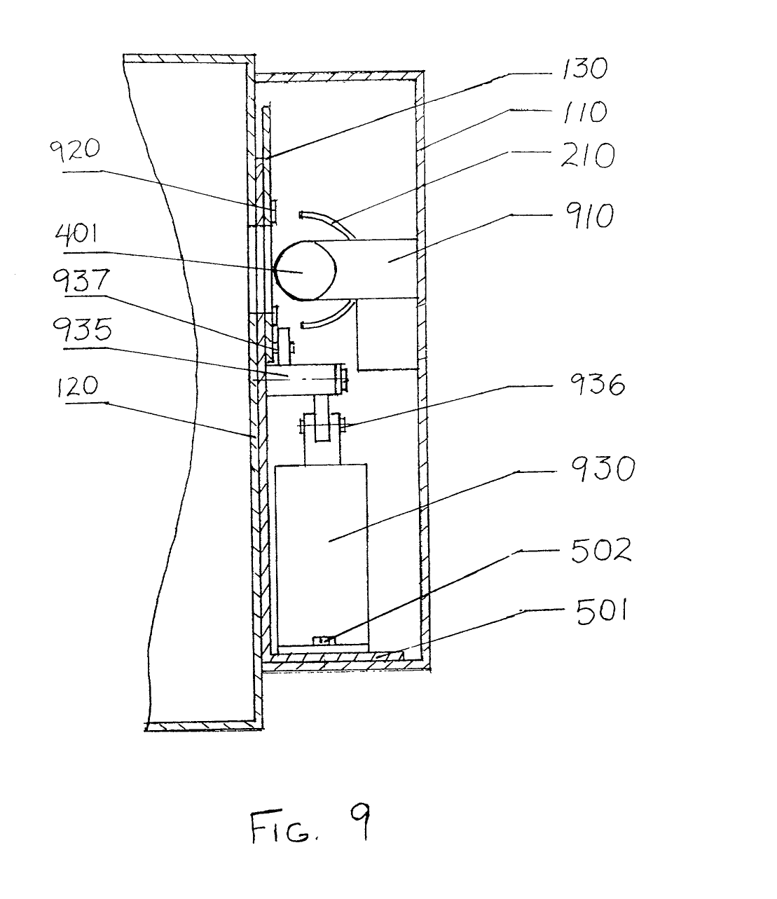

For implementation in an appliances such as a microwave ovens, dishwashers, laundry machines, etc., existing circuit controls, timers, power inputs, and alarm features are adapted for use with the sterilizer features. However, FIG. 8 is a schematic diagram for a circuit 800 to operate a sterilization apparatus according to the present invention.

Circuit board 810 houses a circuit for providing voltage to UV lamp 401 via magnetic leakage converter 550. Voltage is provided to circuit 800 through switch 820 that may, for example, be a safety door switch enabling sterilization only when a door is closed. Timer delay switch 830 allows the circuit to operate for a predetermined period of time specified by a timer input unit (not shown). When circuit 800 is operating, i.e., during a sterilization process, indicator 840 is illuminated to indicate sterilization is being performed. Where appropriate, circuit 800 is also adapted to provide power to the movable components (e.g., exposure panel 130, relay mechanism 515) during circuit operation.

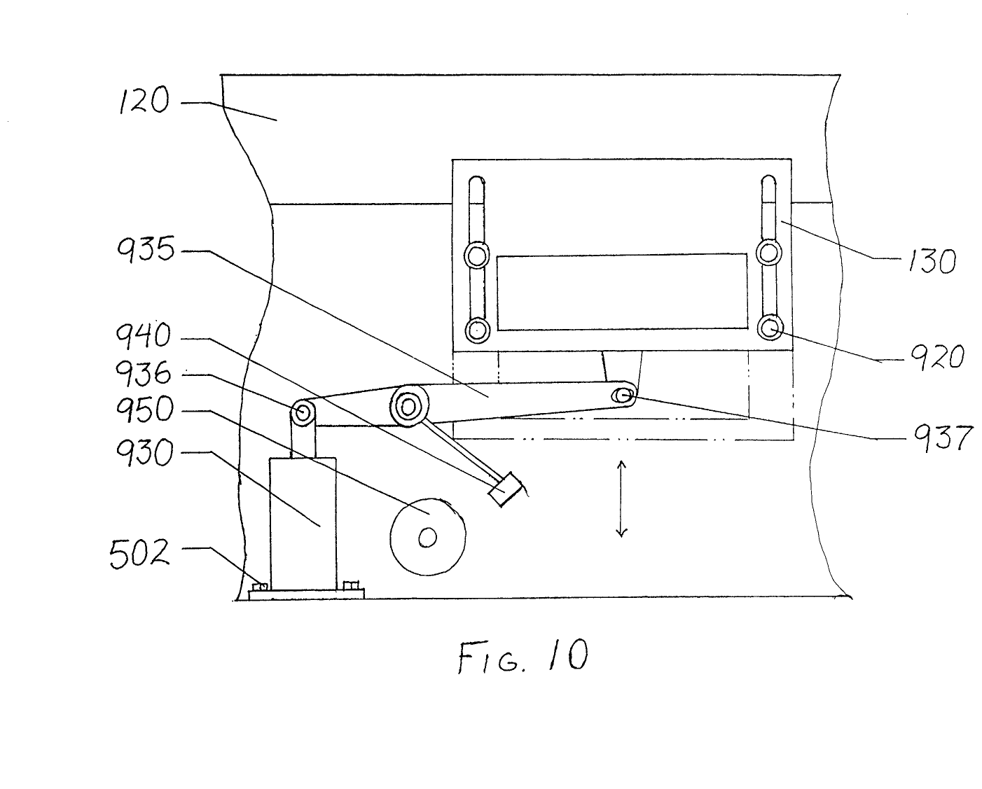

FIGS. 9-10 illustrate another embodiment of the present invention. Here, UV lamp 401 is mounted in Lamp base 910, adjacent sterilization chamber 120. In this embodiment, exposure panel 130 is a retractable window-configuration (similar to embodiment of FIG. 3), and is retractable along an axis track 920 through reciprocal movement of electromagnet 930 and connection bar 935. Connection bar 935 pivotally attaches on one end, via coupling 936, to electromagnet 930, and pivotally attaches on an opposite end, via coupling 937, to exposure panel 130. In this manner, exposure panel 130 may be open and closed to allow radiation from UV lamp 401 to enter into sterilization chamber 120. Bell hammer 940 may optionally be provided on connection bar 935 in a position to strike alarm bell 950 when exposure panel 130 returns to a closed position, indicating an end of the sterilization process.

The foregoing description is only one electromechanical structure for opening and closing exposure panel 130. However, as described above, the present invention could be implemented using other structures, for example, servomotors, pulleys, relays or other conventional electromechanical systems.

With a sterilization apparatus and method according to the present invention, a dual-function appliance (e.g., microwave oven/sterilizer) may be provided that has the same space inside the appliance chamber as a standard, single function appliance of the same style. Additionally, because the UV lamps are stored outside of the chamber, a lower incidence of UV lamp damage and/or contamination is obtained as well as simplified cleaning of the chamber.

Unless contrary to physical possibility, the methods and apparatuses described herein: (i) may be performed in any sequence and/or combination; and (ii) the components of respective embodiments may be combined in any manner.

Although there have been described preferred embodiments of this novel invention, variations and modifications may occur to others and the embodiments described herein are not limited by the specific disclosure above, but rather should be limited only by the scope of the appended claims and the legal equivalents thereof.

What is claimed is:

1. An apparatus for sterilizing an object comprising:
a housing unit including a chamber for containing the object;
an ultraviolet light source for emitting ultraviolet radiation; and
an ultraviolet light source mounting device operative to move the ultraviolet light source into the chamber to sterilize the object and withdraw the ultraviolet light source from chamber thereafter.

2. The apparatus of claim 1 further comprising a control unit operative to move the light source mounting device into the chamber, activate the ultraviolet light source, and withdraw the ultraviolet light source from the chamber.

3. The apparatus of claim 1 wherein the housing unit is a microwave oven.

4. The apparatus of claim 2, wherein the housing unit is a household appliance selected from the group consisting of dishwasher, laundry dryer, toaster oven, and range top oven.

5. A method for sterilizing an object comprising:
(a) placing an object to be sterilized into a sterilization chamber;
(b) moving an ultraviolet light source into the sterilization chamber in response to activation of a sterilization process;
(c) emitting radiation from the ultraviolet light source; and
(d) removing the ultraviolet light source from the sterilization chamber.

6. The method according to claim 5 wherein said sterilization chamber is a heating chamber of a microwave oven.

7. The method according to claim 5, wherein the object to be sterilized is selected from the group consisting of dishes, silverware, food items, contact lenses, cell phones, pacifiers, baby bottles, toys, razors, dish rags, washcloths toothbrushes, false teeth, tools, and containers.

8. An apparatus for sterilizing an object comprising:
a housing unit including a chamber for containing the object;
at least one ultraviolet light source for emitting ultraviolet radiation during a sterilization process, the at least one ultraviolet light source disposed inside said housing unit and outside said chamber; and
at least one exposure panel disposed in a wall of said chamber proximate said at least one ultraviolet light source, the at least one exposure panel configured to allow the at least one ultraviolet light source to irradiate the object during the sterilization process.

9. The apparatus of claim 8, wherein the at least one exposure panel comprises a fixed window allowing the emitted ultraviolet radiation to pass therethrough.

10. The apparatus of claim 8, wherein the at least one exposure panel comprises a retractable panel operative to open during the sterilization process and close during a period of non-sterilization.

11. The apparatus of claim 8, wherein the at least one exposure panel comprises a flip panel configured to be opened during the sterilization process and closed during a period of non-sterilization to facilitate simplified cleaning of said chamber and protect the at least one ultraviolet light source.

12. The apparatus of claim 8, wherein there are a plurality of exposure panels each of which are disposed in various locations of said chamber and in which surfaces thereof are substantially flush with said wall of said chamber for allowing simplified cleaning of said chamber.

13. The apparatus of claim 8, wherein the housing unit comprises a dual-function household appliance having a function in addition to sterilizing objects.

14. The apparatus of claim 13, wherein the dual-function household appliance is one selected from the group consisting of electric-element ovens, crockpots, dishwashers, laundry machines and refrigerators.

15. A microwave oven adapted for sterilization of an object using ultraviolet light, the microwave oven comprising:
an oven chamber for sterilizing the object;
a sterilization unit comprising at least one ultraviolet light source disposed outside the oven chamber during periods of non-sterilization to enable full utilization of said oven chamber for cooking; and
at least one exposure panel disposed in an interior wall of said oven chamber, wherein the at least one exposure panel is configured to allow the sterilization unit to emit ultraviolet radiation onto the object in response to activation of a sterilization process.

16. The microwave oven adapted for sterilization according to claim 15, further comprising:
a rotatable turntable disposed in said oven chamber for supporting the object, wherein the turntable is configured to rotate during the sterilization process.

17. The microwave oven adapted for sterilization according to claim 15, wherein the at least one exposure panel is a panel selected from the group consisting of fixed panel, retractable panel and flip panel design.

18. The microwave oven adapted for sterilization according to claim 17, further comprising, an alarm unit operative to notify a user when a sterilization process is completed.
